(19) United States
(12) Patent Application Publication
(10) Pub. No.: US 2007/0260094 A1
(43) Pub. Date: Nov. 8, 2007

(54) Method for Producing Polyoxymethylene Dimethyl Ethers

(75) Inventors: Heiner Schelling, Kirchheim (DE); Eckhard Stroefer, Mannheim (DE); Rolf Pinkos, Durkheim (DE); Andrea Haunert, Mannheim (DE); Gerd-Dieter Tebben, Mannheim (DE); Hans Hasse, Kaiserslautern (DE); Sergej Blagov, Stuttgart (DE)

Correspondence Address: OBLON, SPIVAK, MCCLELLAND, MAIER & NEUSTADT, P.C., 1940 DUKE STREET, ALEXANDRIA, VA, 22314, US

(73) Assignee: BASF Aktiengesellschaft, Ludwigshafen (DE)

(21) Appl. No.: 11/575,936

(22) PCT Filed: Oct. 19, 2005
(86) PCT No.: PCT/EP05/11234

§ 371 Date: Mar. 23, 2007

(30) Foreign Application Priority Data
Oct. 25, 2004 (DE) 10 2004 051 814.9
Nov. 4, 2004 (DE) 10 2004 053 839.5

(51) Int. Cl. C07C 43/315

(52) U.S. Cl. 568/600

(57) ABSTRACT

A process for preparing polyoxymethylene dimethyl ether of the formula
H₃CO(CH₂O)ₙCH₃ where n=2-10, in which methylal and trioxane are fed into a reactor and reacted in the presence of an acidic catalyst, wherein the amount of water introduced into the reaction mixture by methylal, trioxane and/or the catalyst is <1% by weight based on the reaction mixture. Preferably, a fraction comprising polyoxymethylene dimethyl ether where n=3 and 4 is obtained by distillation from the reaction mixture, and methylal, trioxane and polyoxymethylene dimethyl ether where n<3 and optionally n>4 are recycled into the reaction.

The invention relates to a process for preparing polyoxymethylene dimethyl ethers. Polyoxymethylene dimethyl ethers constitute a homologous series of the general formula
CH₃O(CH₂O)ₙCH₃
in which n is a number≧1. Like the parent molecule of the homologous series, methylal CH₃O(CH₂O)CH₃ (n=1), the polyoxymethylene dimethyl ethers are acetals. Like methylal, they are prepared by reacting methanol with aqueous formaldehyde in the presence of an acidic catalyst. Like other acetals, they are stable under neutral or alkaline conditions, but are attacked even by dilute acids. Hydrolysis converts them in a first step to hemiacetals and methanol. In a second step, the hemiacetals are hydrolyzed to formaldehyde and methanol.

On the laboratory scale, polyoxymethylene dimethyl ethers are prepared by heating polyoxymethylene glycol or paraformaldehyde with methanol in the presence of traces of sulfuric acid or hydrochloric acid at temperatures of from 150 to 180° C. and reaction times of from 12 to 15 hours. This results in decomposition reactions to form carbon dioxide and to the formation of dimethyl ether. At a paraformaldehyde or polyoxymethylene glycol:methanol ratio of 6:1, polymers where n>100, generally n=300-500, are obtained. The products are washed with sodium sulfite solution and subsequently fractionated by fractional crystallization.

U.S. Pat. No. 2,449,469 describes a process in which methylal is heated with paraformaldehyde or a concentrated formaldehyde solution in the presence of sulfuric acid. This affords polyoxymethylene dimethyl ethers with from 2 to 4 formaldehyde units per molecule.

In recent times, polyoxymethylene dimethyl ethers have gained significance as diesel fuel additives. To reduce smoke and soot formation in the combustion of conventional diesel fuel, oxygen compounds which contain only few, if any, C—C bonds, for example methanol, are added to it. However, such compounds are frequently insoluble in diesel fuel and lower the cetane number and/or the flashpoint of the diesel fuel mixture.

U.S. Pat. No. 5,746,785 describes the preparation of polyoxymethylene dimethyl ethers having a molar mass of from 80 to 350, corresponding to n=1-10, by reaction of 1 par of methylal with 5 parts of paraformaldehyde in the presence of 0.1% by weight of formic acid at a temperature of from 150 to 240° C., or by reaction of 1 part of methanol with 3 parts of paraformaldehyde at a temperature of from 150 to 240° C. The resulting polyoxymethylene dimethyl ethers are added to a diesel fuel in amounts of from 5 to 30% by weight.

U.S. Pat. No. 6,392,102 describes the preparation of polyoxymethylene dimethyl ethers by reacting a starting stream comprising methanol and formaldehyde, which has been obtained by oxidation of dimethyl ether, in the presence of an acidic catalyst and simultaneous removal of the reaction products in a catalytic distillation column. This affords methylal, methanol, water and polyoxymethylene dimethyl ethers.

EP-A 1 070 755 discloses the preparation of polyoxymethylene dimethyl ethers with from 2 to 6 formaldehyde units in the molecule by reaction of methylal with paraformaldehyde in the presence of trifluorosulfonic acid. This forms polyoxymethylene dimethyl ethers where n=2-5 with a selectivity of 94.8%, the dimer (n=2) being obtained to an extent of 49.6%. The resulting polyoxymethylene dimethyl ethers are added to a diesel fuel in amounts of from 4 to 11% by weight.

A disadvantage of the known processes for preparing the lower polyoxymethylene dimethyl ethers (where n=1-10) is that the dimer is obtained to a quite predominant extent. A disadvantage of the processes which start from formaldehyde and methanol is additionally that water is formed as a reaction product and hydrolyzes already formed polyoxymethylene dimethyl ethers in the presence of the acidic catalysts. This forms unstable hemiacetals. The unstable hemiacetals lower the flashpoint of the diesel fuel mixture and thus impair its quality. However, too low a flashpoint of the diesel fuel mixture leads to the specifications laid down by relevant DIN standards no longer being fulfilled. Owing to comparable boiling points, hemiacetals are difficult to remove from polyoxymethylene dimethyl ethers. The dimer formed as the main product has a low boiling point and thus likewise reduces the flashpoint, a result of which it is less suitable as diesel fuel additives.

It is an object of the invention to provide an improved process for preparing polyoxymethylene dimethyl ethers which does not have the disadvantages of the prior art. It is a particular object of the invention to provide a process for preparing polyoxymethylene dimethyl ethers which are particularly suitable as diesel fuel additives. Particularly suitable are the polyoxymethylene dimethyl ethers where n=3 and 4 (trimer, tetramer). It is particular object of the invention to provide a process for preparing polyoxyethylene dimethyl ethers with a particularly high proportion of trimer and tetramer.

The object is achieved by a process for preparing polyoxymethylene dimethyl ethers of the formula
H₃CO(CH₂O)ₙCH₃
where n=2-10,
in which methylal (n=1) and trioxane are fed into a reactor and reacted in the presence of a acidic catalyst, wherein the amount of water introduced into the reaction mixture by methylal, trioxane and/or the catalyst is <1% by weight based on the reaction mixture.

In the reaction of methylal with trioxane to give the polyoxymethylene dimethyl ethers, no water is formed as a by-product. The reaction is carried out generally at a temperature of from 50 to 200° C., preferably from 90 to 150° C., and a pressure of from 1 to 20 bar, preferably from 2 to 10 bar. The molar methylal:trioxane ratio is generally from 0.1 to 10, preferably from 0.5 to 5.

The acidic catalyst may be a homogeneous or heterogeneous acidic catalyst. Suitable acidic catalysts are mineral acids such as substantially anhydrous sulfuric acid, sulfonic acids such as trifluoromethanesulfonic acid and para-toluenesulfonic acid, heteropolyacids, acidic ion exchange resins, zeolites, aluminosilicates, silicon dioxide, aluminum oxide, titanium dioxide and zirconium dioxide. In order to increase their acid strength, oxidic catalysts may be doped with sulfate or phosphate groups, generally in amounts of from 0.05 to 10% by weight. The reaction may be carried out in a stirred tank reactor (CSTR) or a tubular reactor. When a heterogeneous catalyst is used, preference is given to a fixed bed reactor. When a fixed catalyst bed is used, the product mixture may subsequently be contacted with an anion exchange resin in order to obtain a substantially acid-free product mixture.

The total amount of water introduced by methylal and trioxane and by the catalyst is <1% by weight, preferably =0.5% by weight, more preferably <0.2% by weight and in paricular <0.1% by weight, based on the reaction mixture composed of methylal, trioxane and the catalyst. To this end, virtually water-free trioxane and methylal are used, and the amount of water correspondingly introduced, if appropriate, by the catalyst is restricted. The hemiacetals (monoethers) and polyoxymethylene glycols formed by hydrolysis in the presence of water from already formed polyoxymethylene dimethyl ether have a comparable boiling point to the polyoxymethylene dimethyl ethers, which complicates removal of the polyoxymethylene dimethyl ethers from these by-products.

In order to selectively obtain polyoxymethylene dimethyl ethers where n=3 and n=4 (trimer, tetramer), a fraction comprising the trimer and tetramer is removed from the product mixture of the reaction of methylal with trioxane, and unconverted methylal, trioxane and polyoxymethylene dimethyl ether where n<3 age recycled into the acid-catalyzed reaction. In a further embodiment of the process according to the invention, the polyoxymethylene dimethyl ethers where n>4 are additionally also recycled into the reaction. As a result of the recycling, a particularly large amount of trimer and tetramer is obtained.

In particularly preferred embodiment, a first fraction comprising methylal, a second fraction comprising the dimer (n=2) and trioxane, a third fraction comprising the trimer and tetramer (n=3, 4) and a fourth fraction comprising the pentamer and higher homologs (n>4) are obtained from the product mixture of the acid-catalyzed reaction of methylal with trioxane. In this context, it is especially preferred to carry out the separation of the product mixture of the acid-catalyzed reaction of methylal with trioxane in three distillation columns connected in series, the first fraction being removed from the product mixture of the reaction in a first distillation column, the second fraction being removed from the remaining mixture in a second distillation column, and the remaining mixture being separated into the third and the fourth fraction in a third distillation column. In this case, the first distillation column may be operated, for example, at a pressure of from 0.5 to 1.5 bar, the second distillation column, for example, at a pressure of from 0.05 to 1 bar and the third distillation column, for example, at a pressure of from 0.001 to 0.5 bar. Preference is given to recycling the first and the second fraction, more preferably additionally also the fourth fraction, into the reaction.

When a homogeneous catalyst, for example a mineral acid or a sulfonic acid is used, it remains in the fourth fraction and is recycled with it into the acid-catalyzed reaction.

The invention is illustrated in detail below with reference to the drawing.

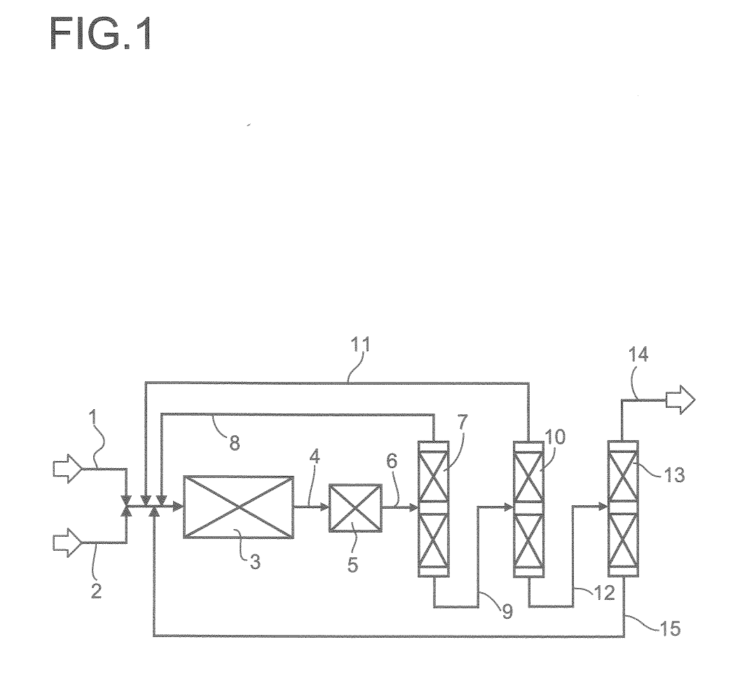

FIG. 1 reproduces a process flow diagram according to one embodiment of the process according to the invention.

A starting stream 1 composed of methylal and a staring stream 2 composed of trioxane are fed together with the recycle streams 8, 11 and 15 into the reactor 3 and reacted there in the presence of a heterogeneous acidic catalyst to give the product mixture 4 which comprises methylal, trioxane and polyoxymethylene dimethyl ether where n=from 2 to 10. The product stream 4 is passed through a bed 5 composed of anion exchange resin to obtain a substantially acid-free product mixture 6. This is fed into a first distillation column 7 in which methylal is removed overhead as a recycle stream 8. The bottom draw 9 of the first column 7 is introduced into a second distillation column 10 in which the dimer (n=2) and trioxane are removed overhead as recycle stream 11. The bottom draw stream 12 of the second distillation column 10 is fed to a third column 13 in which a mixture of trimeric and tetrameric polyoxymethylene dimethyl ether (n=3, 4) is removed overhead. At the column bottom, a recycle stream 15 composed of pentameric and higher polyoxymethylene dimethyl ethers (n>4) is obtained.

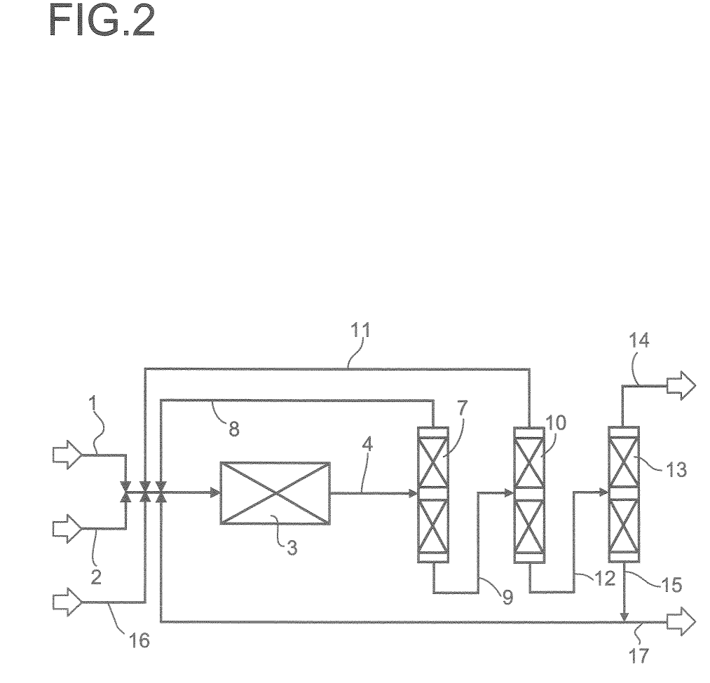

FIG. 2 reproduces the process flow diagram according to a further embodiment of the process according to the invention.

In contrast to the process according to FIG. 1, a homogeneous catalyst is used and is fed into the reactor 3 as a further feed stream 16. A bed composed of anionic ion exchange resin downstream of the reactor 3 is dispensed with and the product stream 4 of the reaction is fed directly to the first distillation column 7. The bottom draw 15 of the third distillation column additionally comprises the homogeneous catalyst. A small stream 17 can be removed from the recycle stream 15 and discharged from the process, in which the catalyst loss can be compensated by the starting stream 16.

### EXAMPLES

### Example 1

30 g of trioxane and 103 g of methylal are heated with 0.2 g of sulfuric acid at 100° C. for 16 hours. After 1, 2, 3, 4, 5, 6, 7, 8 and 16 hours, a sample is taken in each case and analyzed by gas chromatography. After 8 hours, the equilibrium composition had been obtained. This was characterized as follows: 48.7% methylal, 24.5% n=2, 11.7% n=3, 5.2% n=4, remainder n>4.

### Example 2

17 g of trioxane, 30 g of methylal and 15 g of Amberlite® IR 120 ion exchange resin are heated at 100° C. for 24 hours. After 24 hours, a sample is taken and analyzed by gas chromatography. The mixture comprises methylal and polyoxymethylene dimethyl ethers in the following distribution (in % by weight): 70% methylal, 18% n=2, 4% n=3, 0.9% n=4, 4.5% n=5-11, remainder n>11.

### Example 3

85.6 g of paraformaldehyde, 452 g of methylal and 58 g of Amberlite® IR 120 ion exchange resin are heated at 100° C. for 8 hours. After 8 hours, a sample is taken and analyzed by gas chromatography. The product mixture comprises methylal and polyoxymethylene dimethyl ethers in the following distribution (in % by weight): 60.6% methylal, 21.9% n=2, 6.8% n=3, 1.9% n=4 and 0.07% n=5-11.

### Example 4

303 g of trioxane, 1032 g of methylal and 0.2 g of trifluoromethanesulfonic acid are heated at 100° C. for 40 hours. After 40 hours, a sample is taken and analyzed by gas chromatography. The mixture comprises methylal and polyoxymethylene dimethyl ethers in the following distribution (in % by weight): 45.9% methylal, 25.7% n=2, 14% n=3, 7.1% n=4 and 1.4% n=5-11, remainder n>11.

### Example 5

30 g of trioxane, 68.8 g of methylal, 34.4 g of dimer (n=2) and 0.2 g of sulfuric acid are heated at 100° C. for 12 hours. After 12 hours, a sample is taken and analyzed by gas chromatography, The rmixture comprises methylal and polyoxymethylene dimethyl ethers in the following distribution (in % by weight): 33.5% methylal, 23.6% n=2, 15.8% n=3, 9.9% n=4 and 2.6% n=5-11, remainder n>11.

### Example 6

30 g of trioxane, 103.2 g of dimer (n=2) and 0.2 g of sulfuric acid are heated at 100° C. for 12 hours. After 12 hours, a sample is taken analyzed by gas chromatography. The mixture comprises methylal and polyoxymethylene dimethyl ethers in the following distribution (in % by weight): 19.5% methylal, 16.7% n=2, 13.2% n=3, 9.8% n=4 and 4.4% n=5-11, remainder n>11.

### Example 7

30 g of trioxane, 103 g of methylal and 0.2 g of sulfuric acid are heated at 100° C. for 12 hours. After 12 hours, a sample is taken and analyzed by gas chromatography. The mixture comprises methylal and polyoxymethylene dimethyl ethers in the following distribution (in % by weight): 47.8% methylal, 24% n=2, 12.8% n=3, 6.0% n=4 and 0.9% n=5-11, remainder n>11.

As the comparison of example 7 with examples 5 and 6 shows, the recycling of the dimer into the reaction leads to particularly high yields of trimer and tetramer.

1-10. (canceled)

11. A continuous process for preparing polyoxymethylene dimethyl ether of the formula
H₃CO(CH₂O)ₙCH₃
where n=2-10,
in which methylal and trioxane are fed into a reactor and reacted in the presence of an acidic catalyst, wherein the amount of water introduced into the reaction mixture by methylal, trioxane and/or the catalyst is <0.5% by weight based on the reaction mixture, and wherein a fraction comprising polyoxymethylene dimethyl ether where n=3 and 4 is obtained by distillation from the reaction mixture, and methylal, trioxane and polyoxymethylene dimethyl ether where n<3 and optionally n>4 are recycled into the reaction.

12. The process according to claim 11, wherein a first fraction comprising methylal, a second fraction comprising polyoxymethylene dimethyl ether where n=2 and trioxane, a third fraction comprising polyoxymethylene dimethyl ether where n=3 and 4, and a fourth fraction comprising polyoxymethylene dimethyl ether where n>4 are obtained from the reaction mixture.

13. The process according to claim 12, wherein the first fraction is removed from the reaction mixture in a first distillation column, the second fraction is removed from the remaining mixture in a second distillation column, and the remaining mixture is separated into the third and the fourth fraction in a third distillation column.

14. The process according to claim 12, wherein the first and the second fraction are recycled into the reaction.

15. The process according to claim 14, wherein the fourth fraction is recycled into the reaction.

16. The process according to claim 11, wherein the first distillation column is operated at a pressure of from 0.5 to 1.5 bar, the second distillation column at a pressure of from 0.05 to 1 bar and the third distillation column at a pressure of from 0.001 to 0.5 bar.

17. The process according to claim 11 wherein the amount of water introduced into the reaction mixture is <0.2% by weight.

18. The process according to claim 11, wherein the acidic catalyst is a homogeneous or heterogeneous catalyst selected from mineral acids, sulfonic acids, heteropolyacids, acidic ion exchange resins, zeolites, aluminosilicates, silicon dioxide, aluminum oxide, titanium dioxide and zirconium dioxide.

19. The process according to claim 11, wherein the reaction is carried out at a pressure of from 1 to 20 bar and a temperature of from 50 to 200° C.
